(19) United States
(12) Patent Application Publication
(10) Pub. No.: US 2012/0076859 A1
(43) Pub. Date: Mar. 29, 2012

(54) Targeted Lung Delivery of Citrulline and/or Another Nitric Oxide Precursor and a Method for Treatment of Pulmonary Deficiency of Nitric Oxide in Cystic Fibrosis and Other Pulmonary Diseases

(75) Inventor: Thomas Hofmann, Doylestown, PA (US)

(73) Assignee: ACTIVAERO GMBH, Gemunden(Wohra) (DE)

(21) Appl. No.: 12/888,729

(22) Filed: Sep. 23, 2010

(51) Int. Cl. A61K 31/197; A61K 9/14; A61P 11/00; C07C 275/16

(52) U.S. Cl. 424/489; 562/560; 514/564; 428/402

(57) ABSTRACT

A method of treatment of cystic fibrosis and other pulmonary diseases identified by nitric oxide deficiency, comprising administration of a nebulized solution of citrulline and/or another nitric oxide precursor as an inhalable aerosol or an inhalable dry powder for targeted delivery into conducting and central airways. Citrulline or another nitric oxide precursor is formulated as a composition having predetermined limited volume, salinity, pH and osmolality. The composition is nebulized into an aerosol having a mass median aerodynamic diameter (MMAD) between 2 μm and 6 μm.

### BACKGROUND OF THE INVENTION

1. Field of the Invention

The current invention concerns a method of treatment of cystic fibrosis and other pulmonary diseases identified by nitric oxide deficiency. The method comprises administration of a nebulized solution of citrulline and/or another nitric oxide precursor for targeted delivery into conducting and central airways. The invention further concerns a nebulized solution of citrulline or another nitric oxide precursor that is specifically formulated as an inhalable aerosol or an inhalable dry powder.

In particular, the invention concerns a method for targeted delivery of citrulline or another nitric oxide precursor into conducting and central airways wherein said method comprises administration of a nebulizable formulation comprising citrulline and/or another nitric oxide precursor nebulized according to a nebulizing protocol of the invention. The nebulized formulation is deposited predominantly in the target area of conducting and central airways, without any substantial residue of the nitric oxide precursor found in the oropharyngeal area and lower lungs assuring the almost complete delivery of a predetermined amount of citrulline and/or said nitric oxide precursor to the targeted area of the lung without any undesirable secondary symptoms. To that effect, the invention provides an efficacious, safe, nonirritating, physiologically acceptable and compatible inhalable composition suitable for treatment of pulmonary diseases. The most preferred nitric oxide precursor is citrulline.

The citrulline for inhalation is formulated as a composition having predetermined limited volume, salinity, pH and osmolality, that is nebulized into an aerosol having a mass median aerodynamic diameter (MMAD) between 2 μm and 6 μm using a nebulizer able to aerosolize the amino acid solution into particles of required sizes in a time from about 1 to about 3 minutes.

2. Background and Related Disclosures

Certain pulmonary disorders have been found to be associated with deficiency of nitric oxide formation in airways resulting in decreased levels of exhaled nitric oxide while other pulmonary diseases show an increase of exhaled nitric oxide. Among those diseases associated with the decreased levels of nitric oxide are pulmonary diseases such as cystic fibrosis (CF) and primary ciliary dyskinesia (PCD). Other diseases such as, for example, inflammation in asthma show an increase in exhaled nitric oxide. Since the nitric oxide has bronchodilatory effects, it is believed that nitric oxide deficiency may contribute to airway obstruction observed in cystic fibrosis or primary ciliary dyskinesia patients as well as in diseases characterized by arterial pulmonary hypertension.

Nitric oxide is produced in various human tissues and associated with or involved in the regulation of different physiological processes. For example, nitric oxide is known as having a potent vasodilating effect by modulating vascular tone and blood flow and anticoagulation effect by attenuating platelet aggregation and leucocyte adhesion. Its anti-oxidative effect has also been described. Variety of diseases, such as atherosclerosis, hypertension, diabetes, hypercholesterolemia and heart failure are associated with an impairment of endothelium-dependent nitric oxide activity (Cardiovascular Research, 55:250-260 (2002)).

With regard to the pulmonary function, in normal lungs, nitric oxide affects pulmonary vascular tone and resistance and plays an important role in the regulation of vascular, airway and inflammatory events. Consequently, it seems critical that production and regulation of production of nitric oxide is carefully controlled.

In pulmonary diseases, an unregulated deficient production of nitric oxide is associated with pulmonary hypertension and alteration of pulmonary vascular tone while an excessive production of nitric oxide is associated with airway inflammation, vasodilation, hypotension and dysregulated vascular responses. Attempts to provide inhaled nitric oxide to patients with lung diseases provides selective pulmonary vasodilation in a variety of lung diseases associated with hypertension but has a little effect on systemic hemodynamics (Chest, 115:1407-1417 (1999)).

Inhaled nitric oxide has been proposed (J. Appl. Physiol., 84:435-441 (1998)) and tested for its selective pulmonary vasodilating activity. While the nitric oxide inhalation has been shown to reverse pulmonary hypertension without reducing systemic arterial pressure, it has also been suggested that the long-term administration of inhaled nitric oxide may be problematic. The continuous delivery of inhaled nitric oxide requires specifically designed breathing circuits to minimize nitric dioxide formation and to ensure a stable inhaled concentration of nitric oxide making an ambulatory administration unpractical and cumbersome. Additionally, the potential toxicity of nitric oxide is unknown. The article suggests a use of a prodrug (sodium 1-(N,N-diethylamino)diazen-1-ium-1,2-diolate) that spontaneously generates nitric oxide.

Use of inhaled nitric oxide and arginine was proposed (Chest, 126:35-62 (2004)) for treatment of pulmonary arterial hypertension. Inhaled nitric oxide has selective and potent pulmonary vasodilator effecting idiopathic pulmonary arterial hypertension in adults and in children including newborns suffering from pulmonary hypertension and pulmonary arterial hypertension. The article proposes use of arginine supplementation for augmentation of the endogenous production of nitric oxide with oral or infused administration of arginine While arginine infusion resulted in increased plasma levels of L-arginine and L-citrulline, there was a potential drawback of arginine supplementation that includes an increase in the concentration of pro-proliferative polyamines.

Effect of inhaled L-arginine was studied and described in Thorax, 53:172-175 (1998). When the healthy and asthmatic subjects were treated with inhaled L-arginine, L-arginine inhalation increased exhaled nitric oxide in a dose-dependent fashion significantly higher in asthmatic patients then in healthy subjects with a maximum effect observed at 60 minutes. The findings indicate that an increase in availability of L-arginine, the substrate, for nitric oxide synthase leads to an increase in exhaled nitric oxide. The dose-dependent increase in exhaled nitric oxide was found in asthmatic but not in healthy subjects. The results indicate that the production of nitric oxide may be increased with inhaled L-arginine.

Endogenous production of nitric oxide is vital for the decrease in pulmonary vascular resistance that normally occurs after birth. N. Engl. J. Med., 344:1832-1838 (2001) studied a correlation between a low concentration of arginine and presence of persistent pulmonary hypertension in newborns and found that infants with persistent pulmonary hypertension have low plasma concentration of arginine and nitric oxide metabolites. Concentration of citrulline, one of the urea cycle precursors of arginine was also slightly but not significantly lower in the pulmonary hypertension group.

Another disease that is associated with nitric oxide resistance and dysregulated arginine metabolism is sickle cell anemia. JAMA, 294:81-90 (2005) reports that L-arginine is deficient in sickle cell anemia and that the increased arginase activity and dysregulated arginine metabolism contributes to reduced nitric oxide bioavailability and endothelial dysfunction. Authors hypothesize that this is caused by release of erythrocyte arginase which limits arginine availability and releases erythrocyte hemoglobin that scavengers nitric oxide. Distribution of amino acids linked to the L-arginine-nitric pathway, namely ornithine, citrulline and proline was measured. Significant differences in levels of arginine were observed between the control and sickle cell anemia subjects with levels of arginine being significantly lower in sickle cell anemia subjects. Levels of citrulline remained the same. The levels of ornithine were slightly but no significantly different. Levels of proline, on the other hand, were significantly increased in sickle cell anemia subjects.

Over the last decade, several attempts have been made to provide i.v., oral, or pulmonary arginine as a substrate for nitric oxide to patients with cystic fibrosis. Am. J. Respir. Crit. Care Med., 174:208-212 (2006) reports an improvement of pulmonary function in patients with cystic fibrosis with inhaled L-arginine. A single inhalation of L-arginine acutely and transiently improved pulmonary function in cystic fibrosis patients by significantly increasing exhaled nitric oxide concentrations but also resulted in a sustained improvement of FEV₁. Plasma amino acid concentration of L-arginine or L-citrulline did not change significantly over time. However, the concentration of ornithine, the product of L-arginine conversion by arginase, increased significantly. Authors suggest that augmentation of nitric oxide in patients with cystic fibrosis might be beneficial. However, they also suggest that such augmentation may be detrimental in cystic fibrosis.

In diseases such as cystic fibrosis and primary ciliary dyskinesia (PCD), the decreased level of nitric oxide is a reflection and part of the pathophysiology of these pulmonary diseases. Airway nitric oxide concentrations are reduced in cystic fibrosis. Am. J. Respir. Crit. Care Med., 172:1523-1528 (2005) suggest that this is caused by a role of arginase for cystic fibrosis inflammation. The article describes a study of cystic fibrosis patients and their exhaled nitric oxide concentrations, arginase activity in sputum, and their pulmonary function. This study found that sputum arginase activity was significantly higher in cystic fibrosis patients with pulmonary exacerbation compared with patients with stable disease that was higher then arginase activity in healthy subjects. Results indicate the increased arginase activity in cystic fibrosis patient that is commensurate with a degree of disease exacerbation corresponding to the degree of airway inflammation.

The levels of exhaled nitric oxide (FeNO) and nasal nitric oxide (nNO) were measured in chronic airway inflammation in children with primary ciliary dyskinesia (PCD), bronchiectasis, cystic fibrosis and asthma patients and in healthy subjects (Thorax, 57:586-589 (2002). Levels of eNO in PCD and cystic fibrosis were significantly lower than levels observed in bronchiectasis, asthma patients and in healthy subjects.

Since the systemical bioavailability of arginine in cystic fibrosis is known to be reduced, attempts to re-supply arginine orally were made and are described in Eur. Respir. J., 25:62-68 (2005). A single dose of oral L-arginine resulted in significant increase of plasma L-arginine concentration in both the cystic fibrosis and healthy subjects. However, plasma concentration of citrulline, a conversion product of L-arginine by nitric oxide synthase was not increased suggesting that the increase in plasma L-arginine following oral administration did not affect the nitric oxide production.

J. Appl. Physiol., 88:1797-1803 (2000) describes increase of nitric oxide production in isolated lungs of chronically hypoxic newborn pigs by perfusion with L-arginine.

U.S. Patent Application Publication No.: 20010056068 describes the systemic administration of citrulline or citrulline analog in various diseases with systemic nitric oxide deficiency of such diseases as arteriosclerosis, restenosis, hypertension, cardiovascular diseases, preeclampsia, fetal growth and respiratory diseases such as asthma, neonatal respiratory distress syndrome, adult respiratory distress syndrome and pulmonary hypertension. Citrulline or citrulline analog is administered orally, parenterally, enterally, intravaginally or intracervically alone or in combination with other agents enhancing or modulating endogenous nitric oxide production.

PCT Patent Application PCT/GB95/01657 corresponding to the European Patent Application EPI9950924472, discloses the use of a nitric oxide synthase inhibitor, for example L-NMMA, in inhibiting the production of airway mucus secretion in cystic fibrosis, chronic bronchitis and emphysema. The application describes the inhibition of nitric oxide synthase in cystic fibrosis using a formulation comprising the nitric oxide synthase inhibitor, with the goal of reducing the production and secretion of airway mucus.

From the brief description above, it is clear that there is a continuous need for an effective and improved therapy for treatment of lung complications observed in cystic fibrosis, primary ciliary dyskinesia, pulmonary hypertension and other pulmonary diseases. Such therapy would preferably comprise an inhalation of the aerosolized precursor for production of endogenous nitric oxide at a site where the lung complications occur and where the administration of said precursor in form of citrulline, or another nitric oxide precursor formulation would comprise delivering a therapeutically effective amount of citrulline or said precursor directly to the endobronchial space of airways in a shortest possible time.

Although the use of L-arginine as a nitric oxide precursor, an inhibitor of nitric oxide synthesizing enzyme, or systemically administered citrulline or citrulline analog were proposed for enhancement of endogenous nitric oxide production, there are no existing disclosures proposing or implying use of nitric oxide precursors administered by inhalation in a specific manner assuring administration of effective amount of the nitric oxide precursor to the area of the lungs where such administration would achieve a maximizing therapeutic effect. None of the publications described above suggests such an approach and all have certain shortcomings in terms of therapeutic application, route of administration, aerosol production, dose, formulation, etc.

It is therefore an object of this invention to achieve improvement in lung function, increase of production of nitric oxide resulting in increased levels of exhaled nitric oxide, and general improvement of quality of life of patients with cystic fibrosis, primary ciliary dyskinesia and other pulmonary indications characterized with decreased levels of exhaled nitric oxide by administering to a patient in need thereof a therapeutically effective amount of citrulline or another amino acid by inhalation wherein the citrulline or another amino acid are aerosolized in an aerosol having a predetermined particle sizes to be substantially delivered to the conducting and central airways.

All patents, patent applications and references cited herein are hereby incorporated by reference.

### SUMMARY

One aspect of the current invention is a method for treatment of pulmonary diseases identified by nitric oxide deficiency.

Another aspect of the current invention is a method of treatment of cystic fibrosis, primary ciliary dyskinesia, pulmonary arterial hypertension, idiopathic pulmonary fibrosis, acute respiratory disorder syndrome, persistent pulmonary hypertension of the newborn, chronic obstructive pulmonary disease, acute lung injury and other pulmonary diseases wherein said method comprises administration of a nebulized solution of citrulline and/or another nitric oxide precursor, or an analog thereof, formulated for a targeted delivery into conducting and central airways.

Still another aspect of the current invention is a solution of citrulline or another nitric oxide precursor that is specifically formulated and delivered as an inhalable aerosol or an inhalable dry powder by nebulization.

Yet another aspect of the current invention is a solution for inhalation comprising citrulline or another nitric oxide precursor for targeted delivery of citrulline or said precursor into conducting and central airways of the lungs for treatment of cystic fibrosis, primary ciliary dyskinesia, pulmonary arterial hypertension, idiopathic pulmonary fibrosis, acute respiratory disorder syndrome, persistent pulmonary hypertension of the newborn, chronic obstructive pulmonary disease, acute lung injury or another pulmonary disease with decreased exhaled nitric oxide.

Still another aspect of the current invention is a solution for inhalation for delivery of citrulline or another nitric oxide precursor or an analog thereof into conducting and central airways of the lungs, wherein said solution is nebulized into an aerosol with a mass median aerodynamic diameter (MMAD) substantially in the range of about 2 μm to about 6 μm having a substantially monodisperse particle spectrum with a geometric standard deviation (GDS) smaller than 2.2 μm, wherein said solution is nebulized using a suitable electronic, jet or ultrasonic nebulizer optionally used in combination with an airflow control and delivering said nebulized solution by inhalation into the conducting and central airways of a subject suffering from CF or PCD.

Yet another aspect of the current invention is a method for treatment or improvement of pulmonary conditions in cystic fibrosis, primary ciliary dyskinesia, pulmonary arterial hypertension, idiopathic pulmonary fibrosis, acute respiratory disorder syndrome, persistent pulmonary hypertension of the newborn, chronic obstructive pulmonary disease, acute lung injury and other pulmonary diseases characterized with nitric oxide deficiency, by inhalation of a nebulized citrulline solution in a dosage from about 50 mg to about 800 mg/dose into conducting and central airways, said solution nebulized into an aerosol with a MMAD in the range from about 2 μm to about 8 μm.

Still another aspect of the current invention is a formulation comprising from about 50 to about 800 mg, preferably about 200 mg, per one dose, of citrulline salt or an analog thereof, dissolved in a normal or diluted saline solution of from one tenth to a normal strength or in another aqueous solvent containing chloride, wherein said formulation has a pH between 5.5 and 7.0, unbuffered, osmolality between 300 and 700 mOsm/kg, ion concentration between 31 and 300 mM of chloride as a permeant anion and viscosity smaller than 1.5 cp, wherein said formulation is delivered by nebulization in about 1-15 mL, preferably in 3-5 mL of said formulation, using an electronic, jet or ultrasonic nebulizer optionally equipped with airflow control wherein a resulting aerosol has a MMAD between 3 μm and 10 μm and a substantially monodisperse particle spectrum.

Still another aspect of the current invention is a formulation comprising from about 20 to about 1000 mg, preferably about 100-300 mg, per one dose, of a nitric oxide precursor or an analog thereof, dissolved in a aqueous solvent containing chloride or in normal or diluted saline solution of from one tenth to a normal strength, wherein said formulation has a pH between 5.5 and 7.0, unbuffered, osmolality between 300 and 700 mOsm/kg, ion concentration between 31 and 300 mM of chloride as a permeant anion and viscosity smaller than 1.5 cp, wherein said formulation is delivered by nebulization in about 1-15 mL, preferably in 3-5 mL, of said formulation, using an electronic, jet or ultrasonic nebulizer optionally equipped with airflow control wherein the resulting aerosol has a MMAD between 3 μm and 10 μm and a substantially monodisperse particle spectrum.

In yet another aspect of the current invention, citrulline or citrulline analog is administered in a nominal dose between 10 and 50 mg per dose with the corresponding deposited dose in the lung between 5 and 25 mg of citrulline or the citrulline analog.

Still yet another aspect of the current invention is a dry powder formulation comprising from about 50 to 800 mg of citrulline or an analog thereof per one dose, wherein said formulation is milled, spray dried or precipitated into a fine powder with a MMAD between about 3.5 μm and 10 μm and a substantially monodisperse particle spectrum distribution wherein said dry powder formulation is used for inhalation administered from one to four times per day.

Still yet another aspect of the current invention is a dry powder formulation comprising from about 20 to 1000 mg of a nitric oxide precursor or an analog thereof per one dose, wherein said formulation is milled, spray dried or precipitated into a fine powder with a MMAD between about 3.5 μm and 10 μm and a substantially monodisperse particle spectrum distribution wherein said dry powder formulation is used for inhalation.

Still another aspect of the current invention is a two-part reconstitution system comprising citrulline or another nitric oxide precursor in a dry or lyophilized powder form and a diluent stored separately until use.

Yet another aspect of the current invention is the citrulline or another nitric oxide precursor containing solution or dry powder conveniently provided in plastic vials for storage at room temperature.

### Definitions

As used herein:

“(NO)” means nitric oxide.

“(eNO)” means exhaled nitric oxide.

“(NOS)” means nitric oxide synthase.

“(iNOS)” means inducible nitric oxide synthase.

“(iNOS2)” means inducible isoform of nitric oxide synthase.

“Nitric oxide precursor” means any amino acid, salt or analog thereof or any other compound that is involved in urea cycle or another metabolic pathway producing nitric oxide and is or can become a substrate for nitric oxide production. Exemplary amino acids are citrulline, ornithine, proline, glutamine, alanine, and arginine.

“Nitric oxide inhibitor” means compounds, such as, for example, nitro-L-arginine methyl ester (L-NAME), L-N(G) methyl arginine hydrochloride, N-nitro-L-arginine methyl ester, S-(2-aminoethyl)isothiourea and N-(3-(aminomethyl)-benzyl) acetamidine; and L-N⁵(1-iminoethyl)-ornithine, among others.

“Amino acid” means any amino acid that is involved in the nitric oxide pathway or in the urea cycle and is intended to include citrulline, citrulline salt or citrulline analog, ornithine, ornithine salt or analog, proline, proline salt or analog, glutamine, glutamine salt or analog, alanine, alanine salt or analog and arginine, arginine salt or arginine analog.

“(CF)”means cystic fibrosis.

“(PAH)” means pulmonary arterial hypertension.

“(IPF)” means idiopathic pulmonary fibrosis.

“(ARDS)” means acute respiratory disorder syndrome.

“(PPHN)” means persistent pulmonary hypertension of the newborn.

“(PCD)” means primary ciliary dyskinesia.

“(COPD)” means chronic obstructive pulmonary disease.

“(ALI)” means acute lung injury.

“(FEV₁)” means forced expiratory volume defined as the maximum amount of air expired in one second.

“(FeNO)” means fixed exhalation of nitric oxide at fixed flow rate during controlled tidal breathing.

“(MMAD)” means mass median aerodynamic diameter.

“Substantially” means at least 70% and up to 90%.

“Predominantly” means at least 90% and above.

“Active component” means citrulline or another nitric oxide precursor, such as arginine, ornithine, alanine, aspartic acid, proline, glutamate and their salts or analogs.

### DETAILED DESCRIPTION OF THE INVENTION

The current invention concerns generally a method for improvement of pulmonary complications associated with pulmonary diseases that are characterized by nitric oxide deficiency. The method comprises a targeted delivery of nitric oxide amino acids precursors and prodrugs, such as citrulline, citrulline analogs, arginine, arginine analogs, alanine, alanine analogs and ornithine or ornithine analogs and to a certain degree also arginine, arginine salt or analog, formulated and delivered according to the invention. The pulmonary diseases particularly targeted for treatment according to this invention are cystic fibrosis, primary ciliary dyskinesia, pulmonary arterial hypertension, idiopathic pulmonary fibrosis, acute respiratory disorder syndrome, persistent pulmonary hypertension of the newborn, chronic obstructive pulmonary disease, acute lung injury or another pulmonary disease with decreased exhaled nitric oxide.

The method is based on delivering citrulline or another precursor of nitric oxide to a targeted area of the conducting and central lungs where the nitric oxide deficiency is observed. Citrulline or another nitric oxide precursor is nebulized and delivered to the target area as an aerosol having a predetermined particle size having MMAD between 2 and 8 μm in a defined formulation using nebulizers enabling delivery of said aerosol predominantly into the targeted area of the lungs according to the protocol of the invention. Nebulized solution of citrulline or another nitric oxide precursor described herein is formulated and delivered by means assuring a deposition of citrulline or the precursor predominantly in the target area of conducting and central airways, without any substantial residue of citrulline or the precursor found in the oropharyngeal area assuring its almost complete delivery to the targeted area of the lung.

The current invention thus concerns an efficacious, safe, nonirritating and physiologically acceptable and compatible inhalable composition suitable for treatment of cystic fibrosis, primary ciliary dyskinesia or another pulmonary disease, said composition comprising a nitric oxide precursor, preferably citrulline, as an active ingredient. The inhalable composition is formulated for delivery as an inhalable aerosol or as an inhalable dry powder.

I. Nitric Oxide Precursors

The instant invention is based on observations that certain pulmonary diseases are characterized by the deficiency of nitric oxide production. Such deficiency is determined by measurement of exhaled nitric oxide. The invention provides means for increasing the production of nitric oxide in the conductive and central lungs where the nitric oxide deficiency is observed by providing a sufficient amount of nitric oxide precursor, typically a nitric oxide substrate, for inducement of metabolic pathways producing nitric oxide.

The invention provides a means for elevating the exhaled nitric oxide by providing the appropriate substrate to the conductive and central lungs in a form of a nitric oxide precursor. Such elevation will assist in treatment for the pulmonary inflammation observed in cystic fibrosis, primary ciliary dyskinesia and other pulmonary diseases and will help coordinate the impaired/uncoordinated beat of the pulmonary cilia observed in these conditions, thereby improving the mucociliary clearance in cystic fibrosis, primary ciliary dyskinesia and other pulmonary diseases accompanied with a symptomatic lung inflammation.

The invention provides for the aerosol administration of a nitric oxide precursor, preferably an amino acid, and most preferably citrulline, involved in nitric oxide synthesis and metabolism. Aerosol administration according to the invention provides a continuous supply of nitric oxide precursor to the lung. The selected precursor should be directly involved in the urea cycle or in the nitric oxide biopathway and preferably should be different from arginine that has shown undesirable and even toxic effects on the lungs following its aerosol administration alone or, for example, as a salt of therapeutic pulmonary drugs tobramycin or aztreonam.

The aim of the therapy is to increase the level of exhaled nitric oxide with slow supply of the precursor without contributing to the pro-inflammatory and irritating actions of arginine. A substitution of arginine as a precursor and substrate for nitric oxide synthesis in lungs with another precursor eliminates, or at least decreases, the undesirable effects of arginine on the lung tissue and negates the eliminatory effect of arginase and other proteinases on arginine

Most preferred nitric oxide precursor suitable for use as a precursor for nitric oxide according to this invention is citrulline. Citrulline is the amino acid directly involved in the urea and nitric oxide pathway, and therefore acts as the “storage” precursor. In the urea cycle or in nitric oxide pathway, citrulline needs to be metabolized first into arginine, in order to be available as such a substrate. Citrulline, and to the lesser degree also other amino acids involved in the urea cycle are suitable nitric oxide precursors. These precursors are, following the aerosol administration, incorporated into the lung tissue and become participants in the urea cycle and resulting in increased nitric oxide production.

A. Amino Acids Nitric Oxide Precursors

Amino acids are generally used as building block components for protein synthesis, however, many amino acids were also found to assert some specific physiological effects by themselves. For example, amino acids are involved in physiological and biochemical reactions, such as an energy producing Krebs cycle or urea producing cycle that results in production of nitric oxide as a byproduct. Specifically, amino acids involved in urea cycle and in nitric oxide pathway are citrulline, aspartate, arginine and ornithine. Nitric oxide is primarily produced by conversion of arginine to citrulline with nitric oxide synthase, however, while the primary precursors for nitric oxide production are amino acids arginine and citrulline, other amino acid such as ornithine and aspartate are also involved in urea cycle and are therefore, considered to be a nitric oxide precursors. All these amino acid may be advantageously used in practicing the instant invention.

The instant invention provides for delivery of a nitric oxide precursor, preferably the amino acid precursor by inhalation of the aerosolized nitric oxide precursor. Up-to-date, single amino acid have been rarely formulated for and administered by aerosolization. Some attempts were made to administer L-arginine by inhalation, however, these attempts were mostly unsuccessful in that they resulted in pulmonary complications, such as bronchospasm and cough.

I. Arginine

Arginine is an essential amino acid 1-amino-5-(diaminomethylideneamino)pentanoic acid having a formula C₆H₁₄N₄O₂. The molecular weight of arginine is 174.2 g/mol. In the human body, arginine is endogenously produced by the urea cycle and nitric oxide biosynthetic pathway or may be easily supplied externally from animal or plant sources.

In the nitric oxide pathway, L-arginine is the substrate of nitric oxide synthase (NOS) for oxidative reaction resulting in production of nitric oxide and citrulline. In the urea cycle, arginine is synthesized from aspartate and citrulline by the sequential action of the cytosolic enzymes argininosuccinate synthetase (ASS) and argininosuccinate lyase (ASL).

Since certain pulmonary disorders have been found to be associated with deficiency of nitric oxide formation in airways resulting in decreased levels of exhaled nitric oxide it would seem logical to provide patients with these disorders with L-arginine as a precursor of nitric oxide to overcome such deficiency.

To support this premise, it has been reported that L-arginine by itself has certain positive physiological functions. For example, on the positive side, it is known to improve endothelium-dependent vasodilation and its systemic administration has been shown to reduce atherosclerosis and endothelial dysfunction. On the other hand, on the negative side, 20-fold increased plasma concentration of L-arginine was shown to increase plasma insulin, prolactin and glucagon in healthy, diabetic or essential hypertension subjects, thereby resulting in serious hormonal disbalance. Additionally, drugs formulated for and administered by inhalation as arginine salts were shown to cause bronchoconstriction in cystic fibrosis patients and pulmonary inflammation when administered in an aerosol form to the lung in the human subjects and therefore limit the utility of the L-arginine salt, as disclosed in the U.S. Pat. No. 6,660,249, issued on Dec. 9, 2003.

Thus while the administration of arginine by inhalation would seem to be advantageous for patients with lung diseases experiencing decreased level of exhaled nitric oxide, such administration is not without problems as, in addition to the pulmonary complications described above, it may result also in disequlibrating a very sensitive hormonal system in diabetic patients and those suffering from essential hypertension (Cardiovascular research, 55:250-260 (2002)).

Attempts described in Pediatrics, 55:96-100 (1975) to use L-arginine as an aerosolized mucolytic agent in cystic fibrosis patients have been unsuccessful. Authors recommend against use of arginine in cystic fibrosis patients because the inhalation therapy with an arginine solution resulted in cough, airway inflammation and severe deterioration of the patients general conditions.

A reason for these undesirable reactions seems to be that arginine is a substrate for the production of nitric oxide radicals that react with the superoxide anion to form peronitrile. Peronitrile is by itself toxic to the lung tissue and may further react to form highly reactive and toxic hydroxyl radical. Since inflammation is a serious impairment for cystic fibrosis and all other pulmonary diseases which this invention attempts to treat, use of arginine, as known until now, would defeat this purpose as it would worsen rather than better the patient conditions. As a consequence, a better substrate and more controlled, continuous and slower supply of such substrate for nitric oxide production would be desirable.

Additionally, arginine has been found to be an important substrate for immune complex injury in the lung (PNAS, 14:6338-6342 (1991). Since the aerosolization concentrates high levels of the aerosolized arginine in the lung as compared to its dilution seen after intravenous administration, the aerosolization of the arginine would be detrimental rather than advantageous treatment for cystic fibrosis patients or patients suffering from other pulmonary infections.

Although the direct aerosol delivery of arginine to the lung has provided some benefits, such as for example, increase in forced expiratory volume/sec (FEV₁) and in exhaled nitric oxide (Am. J. Respir. Crit. Care Med., 15;174(2):208-212 (2003), several disadvantages appear in conjunction with such aerosolized delivery of arginine.

Arginine has been shown to be pro-inflammatory, and irritating to the airways of the treated subjects. An increased arginase activity can be found in cystic fibrosis patients with this particular activity found in the sputum of cystic fibrosis patients. Such increased activity of arginase not only depletes levels of arginine but it may also contribute to the pro-inflammatory and irritating activity of this amino acid. Additionally, arginine is also a substrate and promoter of bacterial growth, and particularly the substrate for Pseudomonas aeruginosa that is known to cause severe complications for patients with cystic fibrosis and other pulmonary diseases. Besides arginases that specifically metabolize arginine, amino acids in the lungs are also metabolized and eliminated by various proteases and peptidases (Int. J. Biochem. Cell Biol., 40(6-7): 1238-45 (2008)).

It would, therefore, be important to provide a treatment wherein the precursor amino acids would not be metabolized or eliminated by proteases or peptidases or where the precursor would not result in the hormonal disbalance, bronchoconstriction, cough or in another undesirable symptom. On the other hand, arginine, its salt or analog may be used for the purposes of this invention when delivered in smaller concentrations in efficacious manner using previously unknown nebulizing technologies able to deliver arginine or the salt or analog thereof solely to a targeted area of lungs in amounts that eliminate the above described undesirable secondary symptoms.

2. Citrulline for Targeted Lung Delivery

Citrulline is an essential amino acid L-(+)-2-amino-5-ureidovaleric acid having a chemical formula C₆H₁₃N₃0₃. The molecular weight of citrulline is 175 g/mol. Citrulline can be derived from multiple sources and is generally easily commercially available and sold, for example, in a form of citrulline malate, as a performance-enhancing athletic dietary supplement believed to reduce muscle fatigue.

In the human body, citrulline is endogenously synthesized by nitric oxide synthase (NOS) from arginine via high-output nitric oxide pathway or from ornithine via catabolism of proline or glutamine/glutamate via urea cycle. Since these reactions proceed in various cells, the net production of citrulline is highly dependent on cell type and developmental stage.

Citrulline is a key intermediate in the urea cycle, the pathway by which mammals excrete ammonia as well as in the nitric oxide pathway. Two synthetic pathways are connected. In the nitric oxide pathway, arginine is oxidized into N-hydroxyl-arginine, which is further oxidized by nitric oxide synthase (NOS) to nitric oxide. Resulting citrulline is further recycled with cytoplasmic enzymes argininosuccinate synthase (ASS) and argininosuccinate lyase (ASL) into arginine and fumarate and can be again recycled into nitric oxide or, where the resulting arginine is not used for immediate nitric oxide synthesis and enters the urea cycle and is recycled to ornithine and urea using the enzyme arginase. Ornithine, combined with carbamoyl phosphate, is subsequently converted into citrulline by ornithine transcarbamylase. A sufficient amount of citrulline is thus produced by recycling of arginine via nitric oxide pathway or by the urea cycle.

Synthesis of citrulline from arginine occurs at a low level in many cells, and cellular capacity for citrulline synthesis can be markedly increased under circumstances that also induce inducible nitric oxide synthase (iNOS). Thus, citrulline, a coproduct of the reaction catalyzed by nitric oxide synthase (NOS), can be recycled to arginine in a pathway known as the citrulline-nitric oxide or arginine-citrulline pathway. In many cell types, therefore, citrulline can to some degree substitute arginine in promoting synthesis of nitric oxide.

Citrulline, in contrast to arginine, is not used for peptide/protein production, and therefore has a lower propensity for altering metabolism both in the lungs and systemically. In fact, it has been reported that citrulline (although typically a product of arginine metabolism into nitric oxide) can be recycled into arginine (PNAS USA, 87:8612-8616 (1990), which likely also occurs in pulmonary tissue.

Oral citrulline has been used in the treatment of certain urea cycle disorders and intravenous application of citrulline was found to be useful in prematurely born babies to prevent development of persistent pulmonary hypertension of the newborn (J. Thorac. Cardiovasc. Surg., 134(2):319-26 (2007); J. Thorac. Cardiovasc. Surg., 132(1):58-65 (2006); J. Thorac. Cardiovasc. Surg., 134(2):319-26 (2007)). To prevent development of postoperative pulmonary hypertension in children who had congenital heart surgery, the children were treated with an intravenous dose of 150 mg/kg citrulline, along with a maintenance infusion of 9 mg/kg/h of citrulline with a target systemic level of 125 μmol/L.

The US patent application Publication Number 2001/0056068 proposes the systemic intravenous administration of citrulline for possible treatment of various pulmonary conditions, such as acute respiratory disorder syndrome, asthma and chronic obstructive pulmonary disease.

However, the systemically administered citrulline, whether intravenously or orally, does not yield concentrations of citrulline in lungs that would result in increased exhaled nitric oxide or in improvement of pulmonary function, measured by FEV₁. Just the opposite, high or even subtherapeutic doses of citrulline administered systemically might lead to undesirable secondary symptoms such as metabolic changes and increased risk for kidney and gall stones.

The above-identified therapeutic attempts to use systemically administered citrulline have employed solely oral and intravenous routes. A direct pulmonary administration by inhalation has not been disclosed, used or proposed.

The shortcomings observed with systemic administration of citrulline are overcome with the instant invention that provides for the targeted delivery of an aerosolized citrulline to the conductive and central lungs. Aerosolized citrulline, in contrast to arginine, is not be metabolized by arginase, and thus carries much less risk for the arginine toxicity caused by the production of peroxynitrates and lung tissue irritation causing bronchoconstriction, lung tissue inflammation and cough.

The invention provides for administration of citrulline or another nitric oxide precursor by aerosolized route for treatment of pulmonary diseases such as cystic fibrosis, primary ciliary dyskinesia, acute respiratory disorder syndrome, asthma, chronic obstructive pulmonary disease and other pulmonary diseases. Such approach has not been, to inventors knowledge disclosed, used, contemplated or cannot be implied from the known prior art.

Citrulline, as the preferred amino acid, formulated and aerosolized according to the invention will provide clinical benefits for treatment of pulmonary diseases characterized by decreased production of nitric oxide and will be safer than aerosolized arginine.

3. Other Amino Acids

Other amino acids, namely, arginine, ornithin, proline, alanine, glutamine, aspartic acid or their salts or analogs involved in the nitric oxide pathway may be conveniently utilized as substrates and precursors for nitric oxide production similarly to citrulline. With exception of arginine, there are no detrimental effects described or known upon their direct administration to the lungs by nebulization. With regard to arginine, if arginine would be used at all, the doses of arginine would be adjusted to lowest possible effective concentrations and preferably, arginine would be used only in combination with another amino acid in order to avoid its bronchospastic and inflammatory effects on the lungs.

II. Inhalable Compositions for Treatment of Pulmonary Diseases

Citrulline or another nitric oxide precursor disclosed above is formulated as an aerosolizable solution for administration by inhalation. Most preferred nitric oxide precursor is citrulline, citrulline salt or citrulline analog, but the other amino acids, such as ornithine, proline, alanine, glutamine, aspartic acid and arginine, may be administered advantageously to patients suffering from pulmonary diseases via the inhalation route.

Citrulline, as the most preferred amino acid, may be used for aerosolization alone or, alternatively, it may be used in combination in admixture with ornithine, arginine, alanine, glutamine, proline, aspartic acid or with any other precursor or substrate for nitric oxide production when appropriately formulated for such use. Similarly, any other alternative amino acid may be aerosolized alone or in combination with another amino acid. The aerosolized citrulline or another amino acid can be further combined, in a fixed combination, with a nitric oxide synthase inhibitor, such as, for example, L-NAME, L-N(G) methyl arginine hydrochloride, N-nitro-L-arginine methyl ester, S-(2-aminoethyl)isothiourea, N-(3-(aminomethyl)-benzyl)acetamidine, L-N-(1-iminoethyl)-ornithine, among others.

Citrulline or another nitric oxide precursor or substrate is formulated for most efficacious but safe delivery of aerosolized citrulline (or alternative nitric oxide precursor) to the lungs conducting and central airways. In order for inhalation of citrulline or another precursor to be effective for treatment of cystic fibrosis or other pulmonary diseases, citrulline or the precursor must be formulated as an inhalable solution. The inhalable solution containing citrulline and/or the nitric oxide precursor must have certain predetermined properties. These properties include certain pH range, osmolality, viscosity, volume and concentration of the active component. Additionally, the solution must be safe and well tolerated by patients and its delivery must be reasonably fast and efficacious. This invention meets all these requirements and provides an efficacious and well tolerated inhalation treatment for variety of pulmonary diseases.

A. Aerosol or Dry Powder Formulations

Citrulline or another nitric oxide precursor is formulated as an aerosol solution for nebulization or as a dry powder.

a. Aerosol Formulations

The aerosol formulation for nebulization of citrulline or another nitric oxide precursor for treatment and tolerance in pulmonary diseases has certain requirements. These requirements include salinity, osmolality, acidity, ion concentration and viscosity of the nebulization solution.

1. Nitric Oxide Precursor Aerosol Formulation—Effect of pH

The pH of the nebulized formulation containing citrulline or another nitric oxide precursor is an important feature for treatment and treatment tolerance in pulmonary diseases. The pH of the formulation must be maintained as close as possible to the neutral pH range. This range is achieved and maintained with biologically acceptable buffers.

The control of pH of the amino acid formulation is necessary for efficacious delivery of the nebulized citrulline or nitric oxide precursor. When the aerosol is either too acidic or too basic, that is, when the pH it is outside of the safe range of pH from 5.5 to 7, it can cause bronchospasm in central and conducting airways and exacerbate the cough. Any aerosol with the pH of less than 4.5 and above 8.0 typically induces bronchospasm. Aerosols with the pH between 4.5 and 5.5 and between 7.5 and 8 may occasionally cause bronchospasm and typically will also cause inflammation and provoke cough.

Therefore, to avoid development of bronchospasm, cough or inflammation in pulmonary patients, the optimum pH for the amino acid aerosol formulation is determined to be between pH 5.5 and pH 7.0 with tolerable pH between pH 5.0 and 7.5. Consequently the aerosol formulation is adjusted to pH between 5.5 and 7.0 with preferred pH range from about 5.5 to 6.5. Most preferred pH range is from 5.5 to 6.

The pH range of the formulation comprising citrulline or nitric oxide precursor is restricted to a range from pH 5.5 to 7.0, preferably between pH 5.5 and 6.5 and most preferably between pH 5.5 and 6.0.

2. Nitric Oxide Precursor Aerosol Formulation—Effect of Salinity

Salinity of the formulation is another important aspect of this invention. A certain amount of the chloride anion is needed for successful and efficacious delivery of aerosolized citrulline or nitric oxide precursor to a target area. However, because of the sensitivity of the lung tissue and because the aim of this invention is to deliver the aerosol to the specific target area of the lung, this amount is much more specific than amounts provided and typically used for aerosols of other compounds.

Bronchospasm and cough may be sufficiently controlled and/or suppressed when the salinity and osmolality of the solution are in certain limited ranges. Osmolality of the solution is achieved and can be adjusted with a chloride or another anion. Consequently, the preferred solution for nebulization of citrulline or nitric oxide precursor suitable for treatment of pulmonary diseases, including a persistent and severe cough that is safe and tolerated has a range of chloride concentration of between 31 mM and 300 mM providing osmolality of the solution between 300 and 700 mOsm/kg. The given osmolality controls bronchospasm and the chloride concentration, as a permeant anion, contributes to the control of cough.

Salinity of the solution for nebulization containing citrulline or the nitric oxide precursor may be advantageously achieved by using a normal or diluted saline containing from 0.01 to 0.9% NaCl.

The chloride concentration of the formulation is optimally between 31 and 300 mM.

3. Nitric Oxide Precursor Aerosol Formulation—Effect of Osmolality

The osmolality of an aerosolized solution is another important aspect of the aerosol formulation. Osmolality is directly related to the initiation of bronchoconstriction during inhalation. Bronchospasm and cough are regularly induced by inhalation of solutions with osmolality lower then 100 or higher 1100 mOsm/kg. The optimal osmolality is therefore between 300 and 700 mOsm/kg.

During nebulization in a conventional jet nebulizer, osmolality of the solution is known to increase 11% to 62%, as compared with the pre-nebulization value. The peak increase in osmolality is typically observed between 10 and 15 minutes of nebulization and is due to a continuous increase in the concentration of the solute in the nebulizing solution remaining in the nebulizer. Such increased osmolality results in hypertonicity. The reduced time of nebulization to between 1 and 5 minutes, preferably 1-3 minutes, is therefore also important for maintaining the proper osmolality between 300 and 700 mOsm/kg.

In one aspect, exception can be made in cases where the increased sputum expectoration (via high osmotic challenge) is desired from the formulation. In such cases the nebulized solution can be brought to 1000 to 1200 mOsm/kg by adding sodium chloride.

In another aspect, when the airway hydration is desired, hypertonic saline can be effectively combined with aerosolized citrulline or other precursor, by simply adding sodium chloride or other compound to increase osmolality up to approximately 1200 mOsm/kg.

Osmolality of the aerosol solution is optimally, during nebulization, maintained at osmolality between 300 and 700 mOsm/kg, preferably between 450 and 600 mOsm/kg.

4. Nitric Oxide Precursor Aerosol Formulation—Ion Concentration

Ion concentration and permeability are also important for optimalization of the aerosol formulation. In this aspect, chloride ion, osmolality and the presence or absence of a permeant anion and its concentration are connected.

The absence of permeant anion in nebulized solutions is a stimulus for cough even under isoosmolar conditions, and the amount of cough is directly proportional to the concentration of permeant anion. Therefore, not only is the ion concentration important for airway tolerability, but the type of the ion present must also be considered. Inhalation of a solution with osmolality between 225 and 616 mOsm/kg induces cough when the chloride concentration is less than 31 mM. Chloride is the most preferred permeant ion because its presence mitigates some of the adverse effects caused by the hypertonicity of nebulized solutions. A chloride concentration between 31 and 300 mM was found to be optimal.

Chloride anion may be substituted with a selected alternative anion. Examples of salts for the amino acid formulation that produce suitable permeant anion and can be thus used as a substitute of the sodium chloride are calcium chloride, choline chloride, lysine monohydrochloride, potassium chloride, sodium chloride, sodium bromide and sodium iodide. Alternate anion should freely permeate the respiratory mucosa.

5. Nitric Oxide Precursor Aerosol Formulation—Viscosity

Viscosity of the aerosol solution is another important aspect of the current formulation because the rate of nebulization and particle size distribution is directly proportional to the viscosity of the solution. When the rate of nebulization and particle size decrease, the viscosity increases.

The viscosity of the aerosol containing citrulline or another nitric oxide precursor for nebulization for inhalation is set to the optimal level of or near 1.5 cp.

6. Nitric Oxide Precursor Aerosol Formulation—Buffers

The aerosol formulation of the citrulline or nitric oxide precursor must be formulated and also maintained at the neutral range pH. This feature is best accomplished with use of biologically acceptable buffers.

Citrulline and typically also other nitric oxide precursors are weak acids and, therefore, they need to be neutralized with buffers to bring the pH as close to the neutral pH as possible and definitely within pH5.5 and 7.0. Therefore, citrulline or the precursor is neutralized for nebulization with a buffer, such as, for example, commonly used sulfate, carbonate or phosphate buffered saline. Other buffers used in biology are also acceptable. Exemplary biologically acceptable buffers are: 3-{tris(hydroxymethyl)methyl]amino}propanesulfonic acid, N,N-bis(2-hydroxyethyl)glycine, tris(hydroxymethyl)methylamine, N-tris(hydroxymethyl)methylglycine, 4-2-hydroxyethyl-1-piperazineethanesulfonic acid, 2-{[tris(hydroxymethyl)methyl]amino}ethanesulfonic acid, 3-(N-morpholino)propanesulfonic acid, piperazine-N,N′-bis(2-ethanesulfonic acid), dimethylarsinic acid and saline sodium citrate and 2-(N-morpholino)ethanesulfonic acid.

Alternatively, to maintain the neutral range, citrulline can be present as a salt, such as for example, citrulline malate or citrulline hydrochloride. Other precursor may similarly be present in the form of a pharmaceutically acceptable salt.

7. Nitric Oxide Precursor Aerosol Formulation—Additives

There are typically no additives added to the citrulline or nitric oxide precursor formulation for nebulization. Such solution for inhalation is preservative free and preferably no other additives are used. If any additives will be added, they will be physiologically and pharmaceutically acceptable, non-toxic and safe for pulmonary use.

8. Preferred Formulations

In preferred formulations, citrulline or another precursor of nitric oxide may be formulated as a solution, typically buffered solution or as a salt adjusted to neutral pH and/or altered according to the needs of the specific formulation. Generally, the liquid formulation does not require salt formulation. For a dry lyophilized form, citrulline needs to be in a salt form that is reconstituted, prior to aerosolization, with water, saline, or buffered solution.

One preferred formulation for aerosolized citrulline comprises citrulline dissolved in a minimal volume of about 1 to about 10 ml of normal saline containing 0.9% of sodium chloride. A pH of the solution is adjusted to pH between about 5.0 and about 7.5. Osmolality of the solution is adjusted to between about 300 and 700 mOsm/kg. The solution is nebulized into an aerosol having a mass median aerodynamic diameter (MMAD) between 3 μm to 8 μm. A suitable nebulizer is the electronic nebulizer. The citrulline solution is aerosolized for from 1 to about 3 minutes substantially into particles having MMAD between 3 and 8 μm.

Another preferred formulation for aerosolized citrulline comprises citrulline dissolved in a volume of about 4 to about 6 ml of normal or diluted saline. The saline may be diluted to contain from 0.01 to 0.9% of sodium chloride. A pH of the solution is adjusted to pH between about 5.5 and 6.5. Osmolality of the solution is adjusted to between 450 and 600 mOsm/kg. The solution is nebulized into an aerosol having a mass median aerodynamic diameter (MMAD) between 2 and 6 μm using the electronic nebulizer. Time for nebulization is from about 1 to about 3 minutes.

An alternative nitric oxide precursor is similarly formulated for aerosolization with minor modifications needed to accommodate the alternative amino acid chemistry.

b. Dry Powder Formulations Comprising Citrulline or Nitric Oxide Precursor

An alternative way to deliver inhalable citrulline or another nitric oxide precursor for treatment of cystic fibrosis or another pulmonary disease is to formulate said citrulline or said precursor as a dry inhalable powder. Dry inhalable powder is administered to the connecting and central airways by a dry powder or metered dose inhaler (if feasible as a concentrated liquid).

Requirements for the dry powder formulation are similar to those of the aerosol. The dry powder formulation must have similar properties, that is it must be safe and non-irritating to the lungs, must be as close to neutral pH range as possible and must have certain particle size limited to that of the aerosolized droplet, typically, the powder particle sizes are between 3.5 and 10 μm. The chloride ion in the dry formulation may be substituted with, for example, mannitol, sorbitol or other large sugar.

Typically, the dry powder formulation comprises from about 10 to 300 mg, preferably 40 mg, of citrulline or other nitric oxide precursor as an active component. The formulation has potency, on a mass basis, allowing delivery of a sufficient amount of said active component into a lung target area as a dry powder, using dry powder inhaler or metered dose inhaler.

For delivery of the dry inhalable powder formulation, the amino acid salt is milled, precipitated, spray dried or otherwise processed to such particles that when those particles are emitted from the dry powder inhaler, they form an aerosol with a mass median aerodynamic diameter between about 3.5 μm and 10 μm, preferably from about 4 μm to about 5 μm. Examples of powder processing technologies include, but are not limited to media milling, jet milling, spray drying, lyophilization or particle precipitation techniques. Lyophilization and reconstitution techniques are well known in the art and all suitable techniques are hereby incorporated by reference.

All techniques suitable for preparation of dry inhalable powders and any and all improvements thereof as well as any dry powder inhaler or any other inhaler suitable for delivery of the dry powder are intended to be within the scope of the invention.

III. Nebulizers and Other Devices and Properties Thereof

The composition of the invention, described above, provides a nitric oxide precursor, preferably citrulline, formulated in a solution permitting delivery of a therapeutically efficacious amount of the drug, provided that the aerosol generated by the nebulization meets criteria required for such efficient delivery.

Most nebulizers currently used for delivery of pharmaceutical aerosols provide aerosol having particle ranges between 1 μm and 10 μm. This size range has the best balance of inhalability and ability to transport and distribute the active component evenly throughout the lungs. Within this range, smaller particles tend to deposit deeper in the lungs, middle size particles tend to deposit in the central lungs and larger particles tend to deposit in the mouth and throat. Since the small particles contain much less of the active component because the mass increases as the cube of the diameter, the time to deliver an efficacious dose of drugs to the lungs is much longer with smaller particles. In such a system, a selective targeted delivery of the drug to any specific part of the respiratory system is problematic.

The instant active components, namely citrulline or another nitric oxide precursor, need to be deposited predominantly in the conducting and central lungs. For such deposition into the conducting and central airways, the optimal particle size is approximately 4.5 μm. Therefore, the nebulizers best suitable for delivery of the instant aerosol have to have ability to aerosolize the citrulline or nitric oxide precursor composition into particles that are substantially within the range that is deposited into the conducting and central airways. The most optimal size of particles is between 4.5 and 5.5 μm, with an acceptable range of particle size distribution being between 2 and 6 μm and up to maximum 8 μm. Particles smaller than 2 μm and larger than 8μ would not meet criteria and requirements for the aerosolization of citrulline and nitric oxide precursors.

Consequently, a selection of the nebulizer for practicing this invention is a very important aspect of this invention. Generally, the nebulizers that are suitable for the current delivery are specialty monodisperse nebulizers such as the vibrating mesh nebulizers, preferably in combination with airflow control or jet, electronic or ultrasonic nebulizers. No other nebulizers or technology exists that would be able to deposit high amounts of citrulline or nitric oxide precursor to the conducting and central lungs.

Additional problem arising with use of standard nebulizers is that the most currently available pharmaceutical nebulizers produce polydisperse aerosols. Polydisperse aerosols consist of many particle sizes and consequently, the aerosols that are more polydisperse tend to deposit the particles over a wider region of the respiratory tract with a lesser dose of the drug deposited to the targeted area.

On the other hand, the aerosols produced by, for example, the vibrating mesh nebulizers (e.g. eFlow) is monodisperse producing an aerosol with particle sizes having a geometric standard deviation (GSD) smaller than 1.7. Consequently, the majority of the aerosol particles are of the sizes between 2 and 8 μm, with a substantially large portion, typically between 70 and 90%, of these particles having a MMAD between 4 and 5 micrometer.

The advantage of the monodisperse electronic nebulizers or other vibrating mesh nebulizers is that the particle size distribution can be adjusted and tuned to produce particles predominantly in a range having an optimal size and deliver the drug selectively and as quickly as possible to the target area. The same is true for the airflow control nebulizers, where air flow, duration of inhalation, and breathhold can be chosen individually.

Nebulizers and devices suitable for practicing the current invention clearly have to have certain properties that meet the criteria for efficacious delivery of inhalable citrulline or another nitric oxide precursor alone or in combination with each other or with a nitric oxide inhibitor or with other therapeutically effective compounds or formulation additives, to the central and conducting airways according to the method of this invention.

The suitable nebulizers and devices include jet, ultrasonic, electronic, vibrating mesh or vibrating membrane nebulizer, dry powder inhaler, meter dose inhaler, or AKITA® nebulizing systems with or without overpressure. The substantial advantage of these nebulizers is the “breath control” (guided breathing), in which higher doses are delivered to the lung, with less variability. Each of these nebulizers is able to deliver the aerosolable composition substantially into the conducting and central lungs.

An important aspect of the invention is a proper combination of a composition containing citrulline or nitric oxide precursor formulated for aerosolization with a nebulizer able to produce an aerosol with a substantially monodisperse particle spectrum. Such combination assures and permits a delivery of a substantially whole dose of citrulline or said precursor into conducting and central airways without any substantial deposition of the citrulline or amino acid into the oropharyngeal space, or into the lower lungs. Although neither is toxic to the lower lungs or to upper respiratory area, the deposition of the citrulline or the precursor outside of the target area would result in a loss of the active component thereby diminishing its deposition in the lung tissue and a desired impact on nitric oxide production. Additionally, if the large amount of the active compound is delivered to lower lungs, it could easily enter the systemic circulation and may even cause undesirable side effects in terms of changing the cellular metabolism or hormonal balance.

In this regard, aerosolization of citrulline and/or other amino acid advantageously utilizes vibrating mesh nebulizers that produce monodisperse particle sizes, preferably along with specific airflow control and control of breathing pattern of the patient. This arrangement is shown to deposit sufficient amounts of citrulline or another nitric oxide precursor into the conducting and central area of the lung and to achieve aerosolization of the citrulline solution into substantially monodisperse particle sizes with a geometric standard deviation (GSD), between about 1.6 to 2 μm. This can be achieved by using the vibrating mesh nebulizers combined with the airflow control achieved by the nebulizer system known as AKITA® 1 and AKITA® 2, available from Activaero GmbH, Gemünden (Wohra), Germany.

Additionally, other suitable devices for practicing the current invention are handheld breath and airflow control devices embracing AKITA® nebulizer principles. These devices are miniaturized for use as handheld ambulatory devices. Among these devices are, for example, Fox-POP®, Medspray™ and Telemaq™ handheld nebulizers either commercially available from Activaero GmbH, Gemünden (Wohra), Germany or are disclosed in U.S. patent application Ser. No. 12/183747, filed on Jul. 31, 2008, publication number 2009/0056708, herein incorporated by reference in its entirety. Another suitable minidevice is Medspray disclosed in WO 2006/094796, hereby incorporated by reference in its entirety.

A modified inhalation system may further comprise, as a core element, a circular perforated membrane that may be set to vibrate by a piezo-electric actuator. The vibrating motion of the membrane generates an alternating pressure that forces the nebulizable solution through a microarray of perforation in the membrane thus creating a fine aerosol having defined particle sizes. This system is commercially available from Activaero GmbH, Germany, under the trade name AKITA® 2 APIXNEB Inhalation System.

Another device comprising modifications of the inhalation system that can be used for practicing the current invention is the nebulizer that is triggered by the negative trigger pressure detected by a pressure sensor. This nebulizer comprises a compressor that provides a constant inhalation flow rate of 12 liters/minute during inspiration and has a controlled flow, volume and nebulization timing. The system is commercially available from Activaero GmbH, Germany, under the trade name AKITA JET® Inhalation System.

While not required, the nebulization system may optionally include a device for control of breathing pattern. The inhalation system comprises a compressor-driven jet nebulizer that controls the patient's breathing pattern during the inspiration phase and assures a deposition of the aerosol into the central and conducting lungs. During the inhalation, the system controls the number of breaths, the flow rate and inspirational volume. This ability to control these three parameters assures that the patient is given a correct dose.

Some other nebulizing systems suitable to be used in the current invention and commercially available from Activaero GmbH, Germany, under the trade name AKITA® Inhalation System are disclosed in the U.S. Pat. Nos. 6,606,989; 6,463,929; 6,571,791; 6,401,710; 6,681,762; and 7,077,125, or in published applications 2006/0201499 A1 and 2007/0006883 A1, all herein incorporated by reference in their entirety.

Another type of device suitable for practicing the current invention is a breath actuated nebulizer. This nebulizer is characterized by a passive flow and active volume control. Typically, it comprises a single use aerosol generator and a multi-use control device.

The device consists of an inhaler that is connected with a control unit. Inhaler itself is connected with nebulizer where the inhalable citrulline or another amino acid, alone or in combination, or further in combination with a nitric oxide inhibitor is nebulized into predetermined particles having sizes predominantly in the range from about 2 to about 6 μm, MMAD, using an aerosol generator. The filling volume of the nebulizer is approximately 2-4 ml, but can be substantially less, if the aerosol can be concentrated at airway-tolerated formulations. The aerosol generator is activated by pressure detection and is only activated during inspiration phase when the patient is inhaling the aerosolized citrulline or nitric oxide precursor containing composition. The pressure detection is controlled electronically.

This device may be further equipped with means to permit administration of particle-free air, to permit the administration of an aerosolized inhalable citrulline or amino acid composition, and to permit the second administration of the particle free air, each for a preselected time and volume, wherein the cumulative time for these three time intervals correspond to one inspiration time. The time for each of the interval corresponds to from about 1 msec to about 10 sec, preferably from about 200 msec to about 5 seconds.

The inhaler has an integrated flow and volume limited to about 15 liters/minute flow at a pressure of about 10 mbar or lower. When the underpressure at the mouthpiece is below 5 mbar, the flow rate is limited by a mechanical valve. The mechanical valve regulates the flow rate by a adjusting the cross section area. The unit is preset to a volume per one breath. One breath is set to be a time when one inspiration and one expiration occurs. After each inspiration time, the inspiration flow is blocked and expiration allowed. The inspiration flow is restored again for the next inspiration time during the next breath. The modified device and method for its use is disclosed in the U.S. application Ser. No. 12/204,037, herein incorporated by reference in its entirety.

With regard to the selected nebulizer, it is to be understood that its use is intended for aerosolized delivery of citrulline and/or nitric oxide precursor to the central and conducting lungs of subjects having impaired pulmonary function, inflammation or cough observed in cystic fibrosis, primary ciliary dyskinesia, pulmonary arterial hypertension, idiopathic pulmonary fibrosis, acute respiratory disorder syndrome, persistent pulmonary hypertension of the newborn, chronic obstructive pulmonary disease, acute lung injury or another pulmonary disease with decreased exhaled nitric oxide.

IV. Efficacy of Targeted Delivery of Citrulline or Nitric Oxide Precursor

A primary requirement of this invention is to efficiently deliver citrulline and/or another nitric oxide precursor, as an active component, to the central and conducting airways in a most efficient and economic way. Delivery of said active component to the lungs is a function of the size distribution of the inhaled aerosol, the delivery system and the active component content of the particles. Consequently, an initial dose of citrulline or nitric oxide precursor present in the composition for aerosolization, the actual deposited dose of citrulline or nitric oxide precursor in the central and conducting lungs, time of nebulization and frequency of the dosing are all important for reaching the best efficacy of the targeted delivery by nebulization.

A. Efficacy—Delivery Time

Delivery time for aerosolization of the entire amount of the active component present in the aerosol plays important role in the efficacy of the targeted delivery. Given the fact that during nebulization, osmolality of the solution may substantially increase by 11-62%, as compared with the pre-nebulization value, it is very important to deliver the entire volume of the initial aerosol in the shortest possible time in order to maintain the osmolality and other parameters of the formulation, as defined above, throughout the whole time of the nebulization. Change in the osmolality is ultimately translated into change of concentration of the active component in the aerosolable solution.

The peak increase in osmolality is typically observed between 10 and 15 minutes of nebulization. This rise in osmolality is due to the fluid shearing in a high velocity stream of dry gas occurring during nebulization. After generation of primary aerosol droplets during nebulization, solute evaporates from the surface of the aerosol droplets to humidify the air thereby increasing the osmolality in the droplets. Approximately 99% of the droplets then return to the reservoir causing a continuous increase in the concentration of the solute in the liquid remaining in the nebulizer and a continuous increase in the osmolality of the aerosol droplets.

Because of this observable increase in osmolality, the nebulization time is aimed to be restricted to no more than 6 minutes, preferably to 1-3 minutes, with absolute upper time limit of 10 minutes. During this time, there is no measurable change in osmolality and thus there is no concentration effect for the amount of the active component delivered to the lungs.

Proper selection and use of vibrating mesh nebulizers or the other similarly equipped electronic or ultrasonic nebulizers results in shortening of the time for nebulization, thereby eliminating or negating the concentration effect observed with other types of nebulizers. When the time of nebulization is restricted to 6 minutes or less and when the proper nebulizer is selected, there is no change in osmolality and no measurable drug concentration occurs during nebulization.

The optimal time limit set for the delivery of the aerosol comprising citrulline or another nitric oxide precursor is most preferably set to be between 1 and 3 minutes, with time of up to 6 minutes being well tolerated and under certain rare circumstances such time could be extended up to 10 minutes. Under no circumstances would the time for delivery be longer than 10 minutes.

B. Efficacy—Citrulline or Other Nitric Oxide Precursor Dose

Each dose present in the aerosolable composition solution contains a minimal yet therapeutically effective amount of citrulline or another nitric oxide precursor as an active component.

Citrulline is present in the aerosolable composition in amount from 50 to 800 mL. The aerosolable volume is typically between 1 and 10 mL, with preferred aerosolable volume being from about 3 to about 6 mL. The aerosolable solution thus may contain from 50 to 800 mg of citrulline, per one dose. The dose is formulated in the smallest possible volume.

Dose per the whole aerosolable volume 1-10 mL is 200 to 800 mg. Dose per one mL is 60 to 80 mg. Dose to be delivered to the lungs is 80 to 320 mg/dose (assuming 40% deposition efficiency with Akita). Maximal dose of citrulline per one dose is 800 mg fill dose/320 mg deposited. Maximal dose of citrulline per day is 2400 mg fill dose/1000 mg deposited. Other nitric oxide precursors, since they are typically also amino acids, have similar ranges.

A total maximum daily administered dose of said active component is therefore between 800 mg to 2400 mg per day administered in one or more doses of 60 to 800 mg per one dose. The total maximum deposited daily amount should typically not exceed about 1000 mg per day.

The amount of the active component that is present in said aerosolable composition is within above given limits for each individual nitric oxide precursor. The resulting composition is in all cases adjusted such that the aerosolable solution has osmolality between 300 and 700 mOsm/Kg, viscosity about 1.5 cp and pH between 5.0 and 7.5 as the critical parameters.

C. Efficacy—Aerosol Volume

The volume of the diluent used for aerosolization of citrulline or another nitric oxide precursor is also important. Typically, the initial volume for nebulization is between 1 and 10 mL, preferably between 2 and 6 mL, and most preferably from 3-5 mL per one dose. The volume depends on solubility of the citrulline or another nitric oxide precursor in the saline or in another solute and the dose is adjusted such that the aerosolized solution delivers a therapeutically effective dose of the active component in the most efficient and expeditious way.

D. Efficacy—Dosing Regimen

For desired effect of raising and maintaining the increased production of nitric oxide in the conducting and central lungs, the dosing regimen requires either daily administration for a time limited to duration of the disease or conditions to be treated, such as in acute lung injury or acute pulmonary hypertension, or daily and chronic administration, particularly in chronic diseases, such as cystic fibrosis and primary ciliary dyskinesia.

Citrulline or other nitric oxide precursor is administered daily and/or chronically 1 to 4 times per day. In the lowest possible delivery efficacy of about 40% from the administered aerosol dose of 200-800 mg, the deposited dose in the conducting and central lungs would result in from 80 mg to 320 mg of the citrulline or another precursor deposited per one dose. The maximal recommended deposited dose in the target area thus would be from 320 mg to about and preferably not exceeding 1000 mg citrulline or another nitric oxide precursor deposited per day.

In order to achieve such deposited doses, the nominal (aerosol device fill dose) dose will need to be 150 mg to 1000 mg. The nominal dose will be smallest with devices that have high deposition efficiency, such as vibrating mesh nebulizers coupled with airflow control.

E. Efficacy—Determination Thereof

Efficacy of the targeted delivery of citrulline or another nitric oxide precursor is measured by the amount of the drug needed for an increase, to normal levels, of exhaled nitric oxide, and by an increase of mucociliary clearance and pulmonary function.

The amount of exhaled nitric oxide is determined by measuring forced expiratory volume per one second (FEV1). In addition, the effect will be measured via determination of FEV1 (spirometry), exacerbation rate, CF quality of life measures (CFQ-R), and exercise capacity.

V. Shelf-Life and Storage

Stability of the formulation is another very important issue for efficacious formulation. If the drug is degraded before nebulization, a smaller amount of the drug is delivered to the lungs thus impairing the efficacy of the treatment. Moreover, degradation of stored active component may generate degradation products that are poorly tolerated by patients. The dry powder or lyophilized formulation for preparation of the solution for inhalation comprising citrulline or another nitric oxide precursor, should have, preferably, at least one year long shelf life.

According to the invention, citrulline or the nitric oxide precursor suitable for aerosolization is preferably formulated in a predetermined lyophilized dosage form of 10 or 200 mg intended for reconstitution as one single dose for aerosolization. Before inhalation therapy, the lyophilized dose is dissolved in a solute, preferably in saline, provided separately in 1-10 mL vial.

The formulation of the active component is prepared aseptically as a lyophilized powder either for dry powder delivery or for reconstitution. In alternative, it can be prepared as a frozen solution, as a liposomal suspension or as microscopic particles. The extended shelf-life provides for easy and reliable storage of the formulation and allows easy reconstitution or use of the amino acid in dry form suitable for aerosolization.

In practice, the citrulline or another nitric oxide precursor for inhalation suitable for aerosolization is preferably provided as two separate components, one containing a dry citrulline or other nitric oxide precursor lyophilizate or powder, or a salt thereof, and a second containing an appropriate diluent such as, from 0.1 to 0.9 N saline, as described above. The solution for inhalation is reconstituted immediately prior to aerosolization and administration to the patient. The two component packaging for storage prevents problems connected with the long-term stability of the active component in aqueous solvents.

In alternative, the liquid form of the highly concentrated citrulline or another nitric oxide precursor may also be conveniently supplied as a ready to use formulation stored and supplied in 1 mL “Blow-fill-Seal” vial, made of plastic polyethylene material, such as low density polyethylene (LDPE). The selected material for such ready to use formulation prevents absorption of the active component onto the plastic walls of the vial, which is a common occurrence with other plastic materials. The 1 mL fill volume of the vial provides an exact amount of active component that is safe and efficacious for the patient convenience provided, of course, that the therapeutically effective amount of the active component can be supplied in 1 mL dilution. In the above described type of vials covered with an aluminum overwrap, citrulline or nitric oxide precursors for inhalation are stable for at least 9 months and 12 months, respectively, at room temperature. There is no loss of strength under these conditions. At accelerated conditions where the vials are exposed to a temperature of 40° C. and 75% relative humidity, all formulations for extended shelf-life will be shown to retain their full activity for at least 6 months and preferably or one year.

VI. Method for Treatment of Pulmonary Diseases

A method for treatment of the pulmonary diseases characterized with nitric oxide deficiency comprises administration of a nitric oxide precursor including and preferably citrulline as an active component to patients suffering from such pulmonary disease, as a nebulized aerosol comprising said active component, said aerosol having particle sizes of controlled and homogeneous sizes corresponding to sizes of central and conducting airways. The aerosolization is achieved with a suitable nebulizer, preferably electronic, ultrasonic or vibrating mesh nebulizer additionally preferably modified with means to allow a slow and controlled breathing pattern

The method, in practice, provides a nebulized formulation deposited predominantly in the target area of conducting and central airways, without any substantial residue found in the oropharyngeal area or in lower lungs assuring the almost complete delivery of a predetermined amount of citrulline and/or said nitric oxide precursor to the targeted area of the lung without any undesirable secondary symptoms. The method is efficacious and safe for both the acute and chronic treatments. The citrulline or another nitric oxide precursor is formulated as a composition for inhalation having predetermined limited volume, salinity, pH and osmolality, that is nebulized into an aerosol having a mass median aerodynamic diameter (MMAD) between 2 μm and 6 μm using a nebulizer or nebulizer system able to aerosolize the amino acid solution into particles of required sizes in a time from about 1 to about 3 minutes.

An aerosolized solution comprising citrulline or another nitric oxide precursor is administered in a daily dose from about 50 mg to 800 mg dissolved in a saline and nebulized into an aerosol having a mass median aerodynamic diameter 2 micrometer to 6 micrometer and a geometric standard deviation less than 2 using an efficient ultrasonic, electronic or another suitable nebulizer with or without airflow control.

A. Candidate Diseases for Treatment

Candidate diseases for treatment according to the invention are pulmonary diseases characterized with nitric oxide deficiency. Specifically, the method is useful for treatment of cystic fibrosis (CF), pulmonary arterial hypertension (PAH), idiopathic pulmonary fibrosis (IPF), acute respiratory disorder syndrome (ARDS), persistent pulmonary hypertension of the newborn (PPHN), primary ciliary dyskinesia (PCD), compensatory obstructive pulmonary disease (COPD), acute lung injury (ALI), sarcoidosis and asthma, among others.

The possible areas of benefit for this invention are multifold. In general, all pulmonary conditions with nitric oxide deficiency will benefit from increased production of nitric oxide.

1. Cystic Fibrosis

Cystic Fibrosis is an inherited, autosomal recessive disease with chronic pulmonary impairment, and decreased levels of exhaled nitric oxide. The nitric oxide precursor administered to CF patient will improve the mucociliary clearance in cystic fibrosis as well as symptomatic lung inflammation.

As a therapeutic approach, the method of the invention provides nitric oxide precursor, preferably citrulline as an aerosolized solution comprising from 50 to 800 mg of nitric oxide precursor delivered to the conducting and central lungs as an aerosol having MMAD particle sizes preferably substantially within 3-4 μm range. Citrulline is preferred nitric oxide precursor.

2. Primary Ciliary Dyskinesia (PCD)

PCD is represented with upper and lower respiratory tract infection. The disease is characterized by cilia dysfunction. Since the symptoms of PCD, such as rhinitis, otitis media, cough and respiratory distress, are quite common in children, the diagnosis is often made late. The test for screening for PCD includes the measurement of exhaled and nasal nitric oxide.

The nitric oxide precursor administered to PCD patient will help to coordinate the impaired/uncoordinated beat of the pulmonary cilia observed in these conditions and improve the mucociliary clearance in primary ciliary dyskinesia.

As a therapeutic approach, the method of the invention provides nitric oxide precursor, preferably citrulline as an aerosolized solution comprising from 50 to 800 mg of nitric oxide precursor delivered to the conducting and central lungs as an aerosol having MMAD particle sizes preferably substantially within 3-4 μm range. Citrulline is preferred nitric oxide precursor.

3. Pulmonary Arterial Hypertension

Pulmonary Arterial Hypertension (PAH) is a serious condition, in which aerosolized nitric oxide precursor, preferably citrulline, will raise production of nitric oxide that in turn, will contribute to nitric oxide mediated smooth muscle cell relaxation. The mechanism of action here is a vasodilation in the pulmonary vasculature.

As a therapeutic approach, the method of the invention provides nitric oxide precursor, preferably citrulline as an aerosolized solution comprising from 50 to 800 mg of nitric oxide precursor delivered to the conducting and central lungs as an aerosol having MMAD particle sizes preferably substantially within 3-4 μm range. Citrulline is preferred nitric oxide precursor.

4. Primary Pulmonary Hypertension of the Newborn

Similarly, the mechanism of vasodilation is how aerosolized citrulline will benefit children with Primary Pulmonary Hypertension of the Newborn (PPHN).

As a therapeutic approach, the method of the invention provides nitric oxide precursor, preferably citrulline as an aerosolized solution comprising from 50 to 100 mg/day of nitric oxide precursor delivered to the conducting and central lungs as an aerosol having MMAD particle sizes preferably substantially within 3-4 μm range. Citrulline is preferred nitric oxide precursor.

5. Idiopathic Pulmonary Fibrosis

The idiopathic pulmonary fibrosis (IPF) is a serious chronic, progressive form of lung disease characterized by fibrosis of the supporting framework (interstitium) of the lungs. The lung tissue from patients with IPF shows a characteristic set of histologic/pathologic features known as usual interstitial pneumonia. The benefit of aerosolized citrulline for treatment of IPF is mediated via attenuating inflammatory processes, and intraalveolar coagulation that contributes to the pathology in IPF.

As a therapeutic approach, the method of the invention provides nitric oxide precursor, preferably citrulline as an aerosolized solution comprising from 50 to 800 mg of nitric oxide precursor delivered to the conducting and central lungs as an aerosol having MMAD particle sizes preferably substantially within 3-4 μm range. Citrulline is preferred nitric oxide precursor.

6. Acute Respiratory Distress Syndrome

Another possible indication is Acute Respiratory Distress Syndrome (ARDS), along with Acute Lung Injury (ALI), both diseases with complex inflammatory and exsudative changes in the lung, with or without exogenous, noxious triggers. In those diseases, the aerosol application of citrulline (or an alternative nitric oxide precursor) will decrease pulmonary inflammation and reduces accumulation of cells and exsudate into alveolar space, hypoxia and right ventricular dysfunction, with the consequence of improved oxygenation.

As a therapeutic approach, the method of the invention provides nitric oxide precursor, preferably citrulline as an aerosolized solution comprising from 50 to 800 mg of nitric oxide precursor delivered to the conducting and central lungs as an aerosol having MMAD particle sizes preferably substantially within 3-4 μm range. Citrulline is preferred nitric oxide precursor.

7. Acute Lung Injury

Acute lung injury may occur due to accident, surgery, surgery complication, exposure to noxious or toxic gases and toxins, chemical or pharmaceutical intervention or any other cause that disturbs a physiological state of the lungs.

As a therapeutic approach, the method of the invention provides nitric oxide precursor, preferably citrulline as an aerosolized solution comprising from 50 to 800 mg of nitric oxide precursor delivered to the conducting and central lungs as an aerosol having MMAD particle sizes preferably substantially within 3-4 μm range. Citrulline is preferred nitric oxide precursor.

VIII. Combination of Aerosolized Citrulline and Other Nitric Oxide Precursors with Other Therapies

As an alternative strategy, the therapeutic approach for treatment of cystic fibrosis, PCD and other pulmonary diseases with an aerosolized composition comprising citrulline or another nitric oxide precursor may be advantageously combined and/or augmented with other pulmonary therapies. In particular, the aerosolized citrulline or another nitric oxide precursor may be advantageously combined with a beta-agonist, steroid, anti-inflammatory agent, antibiotic, mucolytic or another suitable drug.

Citrulline, for example, provides an additive mechanism to improve the pulmonary condition in cystic fibrosis and may, therefore, be effectively combined with other currently existing and known therapies. Individual therapeutic combinations, doses and specific formulations will depend on the combination of citrulline with the other drug(s). The optimalization of these combinations is based on the knowledge available in the art.

For treatment of cystic fibrosis, for example, the citrulline can be effectively combined and aerosolized in combination with antibiotics, such as aminoglycosides, fluoroquinolones and beta lactam antibiotics; with mucolytics and other cystic fibrosis specific therapies, such as glutathione, N-acetylcysteine and recombinant human DNAse; with surfactant such as tyloxapol, Tween, lucinactant, beractant, calfactant; with purinergic agonists, such as the P2Y2 agonist Denufosol, and amiloride analogs, such as benzamil and phenamil.

Similarly, for other pulmonary diseases, the currently available treatments with therapeutically active drugs, homeopathic treatments, vitamins or other compounds may be advantageously utilized in a combination treatment according to the invention.

All combination therapies suitable for treatments of pulmonary diseases, as defined above, using an aerosolized citrulline or another nitric oxide precursor in such combinations are intended to be within the scope of this invention.

### EXAMPLE 1

### Nitric Oxide Precursor Composition for Inhalation Therapy

This example describes preparation of a composition comprising the nitric oxide precursor citrulline for aerosolization. The composition is prepared in two concentrations, namely comprising 50 and 300 mg of citrulline.

Citrulline (210 or 700 mg) is dissolved in normal (0.9%) saline (3 mL or 10 mL, respectively) (70 mg/mL; citrulline/saline). Osmolality is adjusted with NaCl to osmolality about 450 mOsm/kg, pH is adjusted to pH 6.0 with a phosphate buffer. Concentration of NaCl is 31 mM. Viscosity of the composition is adjusted to 1.5 cp.

Other nitric oxide precursors may be formulated in the same way.

### EXAMPLE 2

### Aerosolization of Nitric Oxide Precursor

This example describes conditions for aerosolization therapy or treatment of pulmonary diseases.

The nitric oxide precursor containing solution for inhalation comprising 50 mg of citrulline or another nitric oxide precursor is prepared according to Example 1. The solution is administered by nebulization into an aerosol having a mass median aerodynamic diameter (MMAD) within a particle size range of 2 μm to 6 μm using an electronic nebulizer with vibrating mesh generating a substantially monodisperse particle spectrum.

Concentration of the exhaled nitric oxide is determined before the administration and following the administration of the aerosolized citrulline, at least in the initial stages of therapy sessions and proper adjustments of the citrulline dosages is made up to 800 mg of citrulline/dose. The dose is adjusted to higher citrulline concentration based on the levels of exhaled nitric oxide.

The other nitric oxide precursors may be similarly formulated, aerosolized and administered with adjustment of the proper dosage based on determination of the exhaled levels of nitric oxide.

### EXAMPLE 3

### Citrulline Solution for Inhalation Used for Treatment of Cystic Fibrosis Patients

This example describes a protocol for clinical trial for treatment of cystic fibrosis patients with inhalable citrulline (50 and 300 mg). The clinical trial is performed in a double blinded, placebo controlled study in approximately 24 patients with cystic fibrosis.

For the study, two solutions containing either 210 mg (3 ml of 7%; 70 mg/mL) citrulline dissolved in normal saline, or 700 mg (10 ml of 7%; 70 mg/mL) citrulline dissolved in non al saline for inhalation, or placebo (3 and 7 mL of isotonic saline having taste masked with quinine) is administered by the electronic nebulizer AKITA® 2. AKITA 2 is vibrating mesh nebulizer equipped with airflow control. The citrulline solution is administered either alone or in conjunction with administration of albuterol twice daily.

Cystic fibrosis patients are selected to be 8 females and 8 males, of predetermined age, having FEV1 40-100% predicted. These patients are enrolled, randomized to three groups, and treated with a single dose of either 210 mg or 700 mg of aerosolized citrulline or with placebo of isotonic saline. The full individual dose of 3 or 10 ml is administered within 6 minutes of treatment time, preferably in 1-3 minutes. The treatment time will not exceed 10 minutes.

Airway irritation and acute bronchospasm will be assessed by measuring spirometry immediately prior to and 30 min post-completion of aerosol administration. A decrease in forced expired volume in one second (FEV1)>20% in the 30 minutes spirometry test will be considered evidence of bronchospasm. All patients will be tested for bronchospasm following the aerosolization in all three groups, and FEV1 will be compared before and after application of citrulline solutions or placebo.

Safety endpoints for determination are: FEV1, systemic (blood) and urine levels of citrulline, arginine, ornithine; taste, GI symptoms and other adverse events.

Efficacy endpoints for determination of efficacy are: pulmonary function (FEV1, measured every two hours), exhaled nitric oxide (NO), FeNO (measured every two hours, change expressed as a percentage of increase, compared to baseline). Mean changes in both FeNO as well as FEV1 will be compared between citrulline doses versus placebo as a primary efficacy analysis.

Exploratory endpoints for determination of efficacy are: sputum expectoration and blue dye technique of measuring nasal mucociliary clearance, and radiolabeled measurement of mucociliary clearance.

What is claimed is:

1. An inhalable composition suitable for the treatment of a pulmonary disease identified by nitric oxide deficiency, wherein said composition is prepared as an inhalable dry powder or as an aerosolizable solution comprising from about 50 to about 800 mg of a nitric oxide precursor selected from the group consisting of citrulline, arginine, ornithine, alanine, proline, aspartic acid, glutamate, salt or analog thereof and wherein said pulmonary disease is cystic fibrosis, primary ciliary dyskinesia, pulmonary arterial hypertension, idiopathic pulmonary fibrosis, acute respiratory disorder syndrome, persistent pulmonary hypertension of the newborn, compensatory obstructive pulmonary disease, acute lung injury, sarcoidosis and asthma.

2. The composition of claim 1 wherein said composition is the aerosolable solution for inhalation wherein said nitric oxide precursor is dissolved in about 3 to 10 ml of buffered solution having adjusted pH between about pH 5.5 and pH 7.0, osmolality between about 300 and 700 mOsm/kg, viscosity to about 1.5 centipoise and permeant anion concentration between 31 and 300 mM.

3. The composition of claim 2 wherein said nitric oxide precursor is citrulline, citrulline salt or analog present in from 50 to 800 mg, dissolved in about 3 to 10 ml of buffered solution having adjusted pH between about pH 5.5 and pH 7.0, osmolality between about 300 and 700 mOsm/kg, viscosity to about 1.5 centipoise and permeant anion concentration between 31 and 300 mM.

4. The composition of claim 3 wherein said buffered solution is a phosphate buffered saline.

5. The composition of claim 4 wherein said pH is adjusted to between pH 5.5 and 6.0.

6. The composition of claim 1 wherein the solution for inhalation comprising nitric oxide precursor is packaged in a sealed low density polyethylene vial under sterile conditions for storage or in a two component packaging comprising a dry or lyophilized nitric oxide precursor salt in one component and a normal or diluted saline in a second component.

7. The composition of claim 6 wherein said solution for inhalation is delivered by a nebulizer in an aerosol having a mass median aerodynamic diameter (MMAD) of particles predominantly in a range between about 2 and 6 μm with a geometric standard deviation (GSD) about 2.2 μm.

8. The composition of claim 7 wherein said nebulizer is an electronic nebulizer comprising a vibrating mesh membrane.

9. The composition of claim 1 wherein the nitric oxide precursor is formulated as a dry powder.

10. The composition of claim 9 wherein the nitric oxide is citrulline, citrulline salt or analog formulated as a dry powder.

11. The composition of claim 10 wherein said dry powder is prepared by milling, dry spraying, lyophilization or precipitation to the particles having a mass median aerodymic diameter from about 3 μm to about 10 μm.

12. The composition of claim 11 wherein the powder has the particles of a mass median aerodymic diameter from about 3.5 μm to about 8 μm.

13. The composition of claim 12 wherein said powder has the particles of a mass median aerodymic diameter from about 4 μm to about 5 μm and additionally comprises an excipient particle wherein said excipient particle is lactose, mannitol, lysine or leucine.

14. The composition of claim 13 wherein said dry powder is delivered by the dry powder inhaler one to four times a day as a dry powder aerosol.

15. The composition of claim 13 wherein said dry powder is delivered by the metered dose inhaler one to four times a day as a dry powder aerosol.

16. A method for targeted delivery of a nitric oxide precursor to the central and conducting airways of the lungs for treatment of pulmonary diseases identified by nitric oxide deficiency, said method comprising steps:
a) preparing an aerosolable solution or dry powder composition for inhalation said composition comprising from about 50 to about 800 mg of the nitric oxide precursor selected from the group consisting of citrulline, arginine, ornithine, alanine, proline, aspartic acid, glutamate, salt or analog thereof;
b) selecting a nebulizer able to generate aerosol of particle sizes substantially between 2.0 and 6.0 μm with geometric standard deviation (GSD) of about 2.0 μm, wherein said nebulizer is an jet, electronic, ultrasonic, vibrating mesh nebulizer, nebulizer equipped with airflow control, dry powder inhaler or meter dose inhaler;
c) nebulizing said aerosolable solution into an aerosol having a mass median aerodynamic diameter of particles substantially between about 2 and 6 μm, having a GSD lower than 2.2 μm;
d) administering said nebulized solution once, twice or several times a day, to a patient for treatment of the pulmonary disease identified by nitric oxide deficiency wherein said disease is cystic fibrosis, primary ciliary dyskinesia, pulmonary arterial hypertension, idiopathic pulmonary fibrosis, acute respiratory disorder syndrome, persistent pulmonary hypertension of the newborn, compensatory obstructive pulmonary disease, acute lung injury, sarcoidosis and asthma.

17. The method of claim 16 wherein said nitric oxide precursor is citrulline formulated as the solution for inhalation, wherein said solution comprises about 50 or about 800 mg of citrulline, citrulline salt or analog, dissolved in about 3 to about 10 ml of a buffered solution.

18. The method of claim 17 wherein said citrulline, citrulline salt or analog is formulated in combination with another nitric oxide precursor selected from the group consisting of ornithine, arginine, alanine, glutamine and, aspartic acid.

19. The method of claim 17, wherein said nitric oxide precursor is formulated in combination with a nitric oxide synthase inhibitor selected from the group consisting of nitro-L-arginine methyl ester (L-NAME), L-N(G) methyl arginine hydrochloride, N-nitro-L-arginine methyl ester, S-(2-aminoethyl)isothiourea and N-(3-(aminomethyl)-benzyl)acetamidine; and L-N⁵(1-iminoethyl)-ornithine.

20. The method of claim 17 wherein said aerosolable solution has pH adjusted to from 5.0 to 7.0 and osmolality from 300 to 700 mOsm/kg.

21. The method of claim 18 wherein said citrulline salt is citrulline hydrochloride or citrulline malate, and wherein said buffered solution is a phosphate buffered saline.

22. The method of claim 18 wherein said nebulizer generates the aerosol having a mass median aerodynamic diameter (MMAD) of said particles is predominantly in a range between about 2 and 5 μm with a geometric standard deviation GSD lower than 2.2μ.

23. The method of claim 22 wherein said nebulizer is an electronic nebulizer with a vibrating mesh membrane either without or combined with an airflow controller.

24. The method of claim 18 used for treatment of a patient with cystic fibrosis or primary ciliary dyskinesia.

25. A method for treatment of cystic fibrosis, primary ciliary dyskinesia, pulmonary arterial hypertension, idiopathic pulmonary fibrosis, acute respiratory disorder syndrome, persistent pulmonary hypertension of the newborn, compensatory obstructive pulmonary disease, acute lung injury, sarcoidosis and asthma wherein said method comprises administering about 6 mL of about 70 mg/mL citrulline, citrulline salt or analog formulated as a solution for inhalation using a vibrating mesh nebulizer, jet nebulizer or ultrasonic nebulizer, dry powder or metered dose inhaler that produces an aerosol with a mass median diameter between about 2 to 6 μm with a geometric standard deviation smaller than 2.2μ, wherein said nebulizer with or without airflow control deposits at least 25% of the total drug in the central and conducting airways of the lungs and wherein said treatment is administered once, twice or several times a day.
